(19) United States
(12) Patent Application Publication
(10) Pub. No.: US 2026/0106036 A1
(43) Pub. Date: Apr. 16, 2026

(54) CLINICAL DECISION SUPPORT SYSTEM AND METHOD

(71) Applicants: TOPCON CORPORATION, Tokyo (JP); Topcon Healthcare, Inc., Oakland, NJ (US)

(72) Inventors: Ali Amin TAFRESHI, San Diego, CA (US); Christopher Kai LEE, Denver, CO (US); Carolin Tanja SEITH, Linkenheim-Hochstetten (DE); Mary Kathryn DURBIN, San Francisco, CA (US); Hanna Kaarina KINNUNEN, Helsinki (FI); Samantha Aliana MROZ, Hatfield, PA (US)

(73) Assignees: TOPCON CORPORATION, Tokyo (JP); Topcon Healthcare, Inc., Oakland, NJ (US)

(21) Appl. No.: 18/916,797

(22) Filed: Oct. 16, 2024

(51) Int. Cl. G16H 50/30; G16H 10/60

(52) U.S. Cl. CPC G16H 50/30; G16H 10/60

(57) ABSTRACT

A clinical decision support system and method allows a user to display, review, and analyze health data. One or more algorithms can be used to analyze health data in order to produce a score. The score is presented graphically along with related examination parameters for review by a user. The criticality of examination parameters is also displayed graphically.

### FIELD OF THE INVENTION

The present disclosure relates generally to analysis and review of data associated with examination parameters, and more particularly to a clinical decision support system and method.

### BACKGROUND

Patients visit health care providers for routine physical examinations and to deal with specific medical issues. The health care provider examines the patient and determines whether they can help the patient in view of the patient's current condition and the patient's past condition. In order to determine a patient's past condition, the health care provider needs to review the patient's records. This can be difficult if the patient's records are not readily available for review. As such, patient health data needs to be easily accessible by multiple entities. In addition, the health care provider may need to determine if the patient should be referred to a specialist. Patients also visit eyeglass stores, ophthalmology clinics, and other entities related to vision and eye care where there is an opportunity to identify issues the patient may have. However, employees of eyeglass stores and ophthalmology clinics may not be able to identify patient issues without assistance. What is needed is a method for capturing, storing, analyzing and sharing patient examination data to assist in patient diagnostics and improving the quality of referrals to specialists.

### SUMMARY

A clinical decision support system and method are described herein that analyze patient data, automate determination of the likelihood that a patient has a disease or condition to support decision making, and provide for convenient display and organization of relevant patient data and analytics. The method includes receiving health data associated with a patient, where the health data includes examination parameters. A score associated with the patient is calculated using an algorithm to analyze the received health data. The algorithm can be selected from a plurality of candidate algorithms and can be an artificial intelligence algorithm. A report is displayed comprising the score and an indication of a relative criticality of each of the examination parameters. In one embodiment, the indication of the relative criticality of each of the examination parameters is based on a normative database. In one embodiment, the indication of the relative criticality of each of the examination parameters is based on thresholds for abnormality. In one embodiment, the relative criticality is with respect to the score. The relative criticality of each of the examination parameters with respect to the score can be indicated by color.

In one embodiment, a second score associated with the patient can be calculated using a second algorithm to analyze the received health data. A second report can be displayed comprising the second score and an indication of the relative criticality of each of the examination parameters with respect to the second score. The report and the second report can be displayed together and an indication of the relative criticality of each of the examination parameters with respect to the score or the second score can be displayed depending upon a user selection. The score and the second score can be consolidated for display in an image.

In one embodiment, user input modifying one of the examination parameters is received and, in response, the score is updated. In one embodiment, user input selecting one of the examination parameters is received and, in response, data related to the selected one of the examination parameters is displayed in response to the user input. In one embodiment, user input requesting scores and examination parameters of other patients having health data similar to the patient is received and, in response, the other patients' scores and examination parameters are displayed.

An apparatus having memory storing computer program instructions for providing a score associated with a patient and a computer readable medium storing instructions for providing a score associated with a patient are also described herein.

### BRIEF DESCRIPTION OF THE DRAWINGS

FIG. 1 shows a clinical decision support system (CDSS) in communication with a plurality of devices according to one embodiment;

FIG. 2 shows the CDSS of FIG. 1 along with its configuration with respect to other hardware and software components;

FIG. 3 shows the CDSS of FIG. 1 according to one embodiment;

FIG. 4 shows a method for operating a CDSS according to one embodiment;

FIG. 5 shows results of a selected algorithm used to analyze health data according to one embodiment;

FIG. 6A shows a score and examination parameters displayed in response to selection of a tab according to one embodiment;

FIG. 6B shows a score and examination parameters displayed in response to selection of a tab according to one embodiment;

FIG. 6C shows a score and examination parameters displayed in response to selection of a tab according to one embodiment;

FIG. 6D shows a display in which a user can select an examination parameter according to one embodiment;

FIG. 6E shows a display having a text box in which a user can enter an examination parameter value according to one embodiment;

FIG. 6F shows how a new value entered as an examination parameter is displayed differently from an original examination parameter value according to one embodiment;

FIG. 6G shows a display having input boxes for allowing a user to enter examination parameters according to one embodiment;

FIG. 6H shows a display shown to a user in response to selection of an examination parameter according to one embodiment;

FIG. 6I shows a display in which a user is selecting to view another patient's data according to one embodiment;

FIG. 6J shows a display in which a user has selected to view comments regarding a patient according to one embodiment;

FIG. 7 shows scores associated with different algorithms displayed consolidated into a single image according to one embodiment;

FIG. 8 shows a report having a gauge-type chart for displaying a score according to one embodiment;

FIG. 9 shows a high-level schematic of a computer for implementing method and systems described herein;

FIG. 10 shows a method for opticians to use AI in order to assist in performing a patient's eye health exam according to one embodiment; and

FIG. 11 shows data flow for image analysis indicating how data flows among a user, data management software, and an AI product according to one embodiment.

### DETAILED DESCRIPTION

Data management software comprises a plurality of modules for acquiring, storing, analyzing, and displaying various patient data. Each of the plurality of modules can be enabled or disabled for a particular entity or user of the data management software. The present disclosure describes a clinical decision support system (CDSS) for glaucoma but the disclosure also supports clinical decision support systems for other types of medical conditions, medical issues, etc. In one embodiment, the CDSS is a module (i.e., a part) of the data management software and is used to estimate a patient's health relative to Glaucoma. The score is based on well-known algorithms. The purpose of the score, in one embodiment, is not to diagnose glaucoma but instead to help a user, such as a primary health care provider, to determine if there is a need for further examination or evaluation. In one embodiment, the algorithms that can be used for risk calculations include, but are not limited to, multi-factoral optical coherence tomography (OCT) screening score (MOS), glaucoma health score (GHS), and ocular hypertension treatment study (OHTS).

In one embodiment, each algorithm uses the patient's examination data to calculate a score that will indicate the likelihood the patient will develop glaucoma. The score can help a user make an informed decision regarding the patient's care and the next steps.

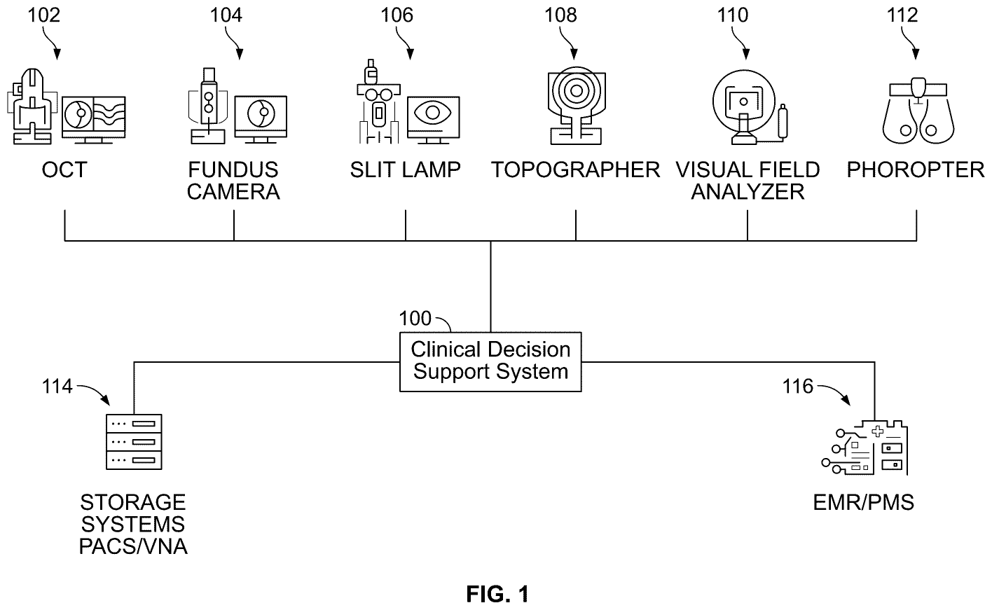

FIG. 1 shows a clinical decision support system (CDSS) 100 in communication with various examination devices including optical coherence tomography (OCT) device 102, fundus camera 104, slit lamp 106, topographer 108, visual field analyzer 110, and phoropter 112, all of which may be used to generate examination parameters during an examination of a patient's eye. CDSS 100 is also in communication with storage systems 114 comprising a picture archiving and communication system (PACS) and a vendor neutral archive (VNA). CDSS 100 is also in communication with electronic medical records (EMR) and practice management software (PMS) 116 which allows a user to access and update data of EMRs. Data received from examination devices 102-112 along with patient data from storage systems 114 and EMR/PMS 116 allows a user accessing clinical decision support system to display, review, and manage patient health data. The CDSS also assists a user in the selection and application of algorithms with respect to patient health data to determine a patient's condition and if that condition requires a referral to another doctor, such as a specialist. CDSS 100 can also be used for gathering and analyzing data in order to promote efficient and accurate decisions regarding a patient's health.

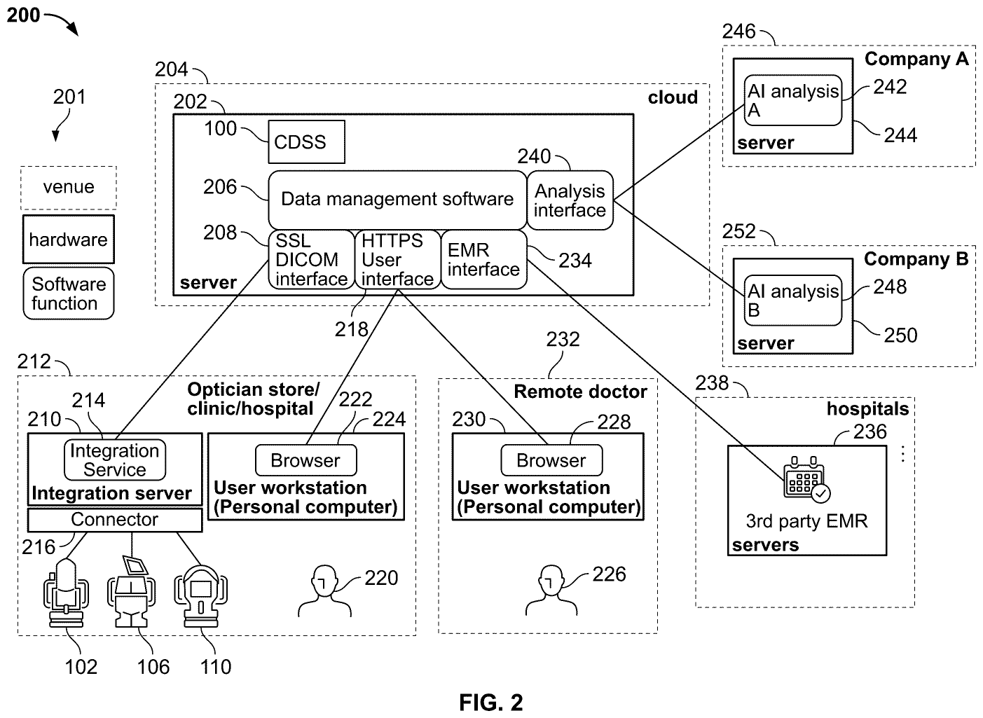

FIG. 2 shows CDSS 100 and its configuration and functionality with respect to other hardware and software in patient health system 200. It should be noted that a venue is identified in FIG. 2 by a dashed line border, hardware is identified by a bold solid line border, and a software function is identified by a thin line border as shown by legend 201. CDSS 100 is implemented on server 202 which is implemented within cloud computing system 204. CDSS 100 is a module of data management software 206 which is also implemented on server 202. Data management software 206 is in communication with secure sockets layer digital imaging and communication in medicine (SSL DICOM) interface 208, HTTPS user interface 218, EMR interface 234, and analysis interface 240 all of which are implemented on server 202. In one embodiment, CDSS 100, SSL DICOM interface 208, HTTPS user interface 218, EMR interface 234, and analysis interface 240 are implemented in data management software 206, however one or more of CDSS 100, SSL DICOM interface 208, HTTPS user interface 218, EMR interface 234, and analysis interface 240 can be implemented on server 202 as standalone modules. SSL DICOM interface 208 is in communication with integration server 210 located at optician store 212 (which alternatively may be a clinic or hospital).

Integration server 210 operates integration service 214 which receives data from devices including OCT device 102, slit lamp 106, and visual field analyzer 110 via connector 216. It should be noted that integration service can receive data from additional devices, such as those shown in FIG. 1, although only three devices are shown in FIG. 2 for clarity.

HTTPS user interface 218 allows user 220 located at optical store 212 to access data using browser 222 operated on user workstation 224 (also referred to as personal computer) which are also located at optical store 212. HTTPS user interface 218 also allows user 226 located at remote doctor location 232 to access data using browser 228 operated on user workstation 230 (e.g., browser 228 and workstation 230 together form a client and the client can include/support other types of software) which are also located at remote doctor location 232.

Electronic medical record (EMR) interface 234 is implemented on server 202 and allows data management software 206 to communicate with a 3^{rd} party EMR service implemented on 3^{rd} party EMR servers 236 operated at hospitals 238. In one embodiment, EMRs are used for data exchange. For example, a remote EMR, such as 3^{rd} party EMR, is in communication with data management software 206 to transmit and received EMRs. EMRs can also be sent to other doctors in order to obtain additional opinions. In one embodiment, patient data is stored in an EMR and that EMR can be modified by various entities, such as users, CDSS 100, etc.

In one embodiment, CDSS 100 has analysis software (e.g., artificial intelligence analysis software) and can analyze images and other patient data using artificial intelligence or other types of analysis software. CDSS 100 can also transmit information to other locations for analysis using artificial intelligence or other types of analysis software. Analysis interface 240 is implemented on server 202 and allows data management software 206 to communicate with company A 246 and company B 252. Company A server 244 implements Artificial Intelligence (AI) analysis algorithm A 242 and is used to analyze health data of a patient using a particular AI algorithm. Similarly, company B server 250 implements AI analysis algorithm B and is used to analyze health data of a patient using a different AI algorithm. It should be noted that although company A 246 and company B 252 are described as implementing AI algorithms, other types of algorithms may be used as well to analyze health data of a patient.

In one embodiment, CDSS 100 is part of data management software 206 and CDSS is opened (i.e., launched) from data management software 206 (note that CDSS 100 is shown separate from data management software 206 in FIG. 2 according to a different embodiment). In one embodiment, examination parameters and/or medical test results are generated using examination equipment and the examination parameters and/or medical test results can be input to CDSS 100 or otherwise made available to CDSS 100. CDSS 100 can be launched when a user meets a patient who qualifies for a glaucoma screening. In one embodiment, a patient qualifies if, for example, they show symptoms or if the screening is part of a routine health assessment. The user then checks (e.g., reviews) the score of an algorithm that is currently active (i.e., selected). Scores of other algorithms can be checked as well. A user can then check examination data (e.g., examination parameters) included in the calculation of scores. A user can change or exclude data where necessary and check the scores again.

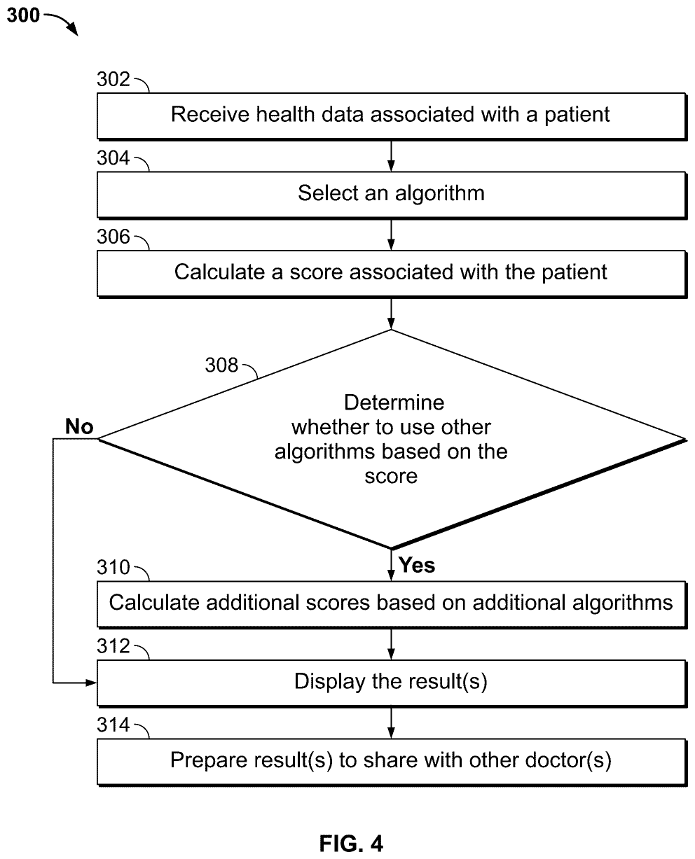

In one embodiment, CDSS 100 cannot be launched before the user is logged into data management software 206. In addition, CDSS 100 must be enabled in data management software 206 and the user must have a valid license to use CDSS 100. The user must also have a particular patient's information to open a clinical viewer and access the patient's examination parameters. A user can then launch CDSS 100 by selecting the glaucoma tab (e.g., glaucoma tab 404 shown in FIG. 4) in data management software 206. CDSS 100 launches and automatically shows a score for the patient immediately, calculated for the active algorithm using the latest examination results. In one embodiment, the active algorithm can be selected based on user or organizational preferences. In one embodiment, CDSS 100 can be launched from within an EMR of a patient.

In one embodiment, CDSS 100 must have at least one algorithm enabled. Depending on configuration, several algorithms may be enabled and available for score calculations. The active algorithm is the one currently selected in an algorithm view. The other algorithms with licenses are shown as individual tabs in one embodiment. They are referred to as the enabled algorithms.

If more than one algorithm is enabled, a user can change any one of the enabled algorithms to be the active algorithm and view the associated score. In one embodiment, each algorithm uses a set of different risk factors to calculate the score. In one embodiment, risk factors are examination parameters which pertain to a specific disease or a specific algorithm used to calculate the risk of the specific disease.

In one embodiment, for each active algorithm, an associated score is presented in numerical format. It should be noted that an associated score can also be presented qualitatively. The score is also visualized with a gauge where different color risk levels aid a user in reviewing the risk estimation. In one embodiment, individual risk factors associated with a score are displayed in addition to a score.

In one embodiment, CDSS 100 will automatically select the latest available data to be used in risk calculations. A user can select examinations and/or examination parameters from a drop-down menu that shows all the available examinations within a time range. In one embodiment, to change the data used in the risk calculations a user can select risk factors for a selected eye (i.e., an eye of a patient that has been examined) and the score value and the gauge graphic are updated based on the selected risk factors. It should be noted that, in one embodiment, fundus and OCT are interconnected. If the active algorithm is changed after changing the examination data, the change of examination data is carried over to the new algorithm. In one embodiment, the algorithm can be selected based on the examination parameters associated with the algorithm. For example, the algorithm can be selected based on age since scores may become more important as a patient ages.

It should be noted that bad or incorrect data can be worse than no data at all. Examinations and/or examination parameters can be left out if a user does not want to include them in the score calculation. In one embodiment, the score is recalculated after the user selects examinations and/or examination parameters that should be left out of the score calculation. If there is not enough data to calculate a score after examinations and/or examination parameters are excluded, a score will not be calculated.

In one embodiment, CDSS 100 requires the use of discrete data storage (DDS) to safely and securely store data.

The term artificial intelligence (AI), as used herein, pertains to any technique that enables computers to mimic human intelligence using logic, if-then rules, decision trees, and/or machine learning (including deep learning). Machine learning as used herein pertains to a subset of AI that includes statistical techniques that enable machines to improve at tasks with experience. Deep learning as used herein pertains to a subset of machine learning comprising algorithms that permit software to perform tasks, such as speech and image recognition, by using multi-layered neural networks to analyze vast amounts of data.

AI can be used to analyze a fundus image in order to recognize diseases such as diabetic retinopathy (DR), age related macular degeneration (AMD), and glaucoma and generate a severity classification. AI can also be used to analyze an OCT scan in order to recognize pathologies and generate an indication of their severity. AI can further be used for multi-modal analysis of images and scans, other eye data such as perimetry or intra-ocular pressure (IOP), and a patient's account of their medical history (i.e., anamnesis). AI can also be used for multi-source analysis and forecasting based on various exam data and history, patient history, normative data, different AIs, etc. AI can produce outcomes including diagnosis, recommendations, and clinical guidance. AI analysis can help opticians and optometrists make informed decisions about which patients to send to an ophthalmologist for review, assist in early detection of diseases, and help both optometrists and ophthalmologists provide more services to patients.

In one embodiment, AI is used in screening as follows. A patient is registered (e.g., an electronic medical record for the patient is generated in practice management software). Data management software 206 identifies the electronic medical record of the patient and creates worklists. Examinations are performed according to the worklists and the examination parameters generated during the examinations are stored in data management software 206. Images are sent for AI based image analysis (e.g., sent to company A 246 and/or company B 252 for analysis). The examination parameters and AI analysis are reviewed at a screening center. Based on the review, a patient may be referred to an ophthalmologist at a reading center. The ophthalmologist at the reading center reviews patient data including the examination parameters and AI analysis and replies to the screening center if needed. The ophthalmologist at the reading center can also refer the patient to an eye care provider. A referral, if needed is sent to the eye care provider along with relevant patient data. The screening center receives diagnosis, recommendations, and follow up instructions. A screening center can be a medical or a non-medical or a non-eye care institution. In one embodiment, the screening center performs data acquisition for screening purposes, which does not require the presence of a physician. The screening itself (evaluation of the acquired data) can be performed at a reading center, where healthcare professionals (ophthalmologists, advanced care physicians) review the acquired data and give recommendations if the patients should be seen by a physician.

Images can be sent for analysis in various ways. In one embodiment, images can be sent for analysis on demand. In this case, a user can select the images to be sent and then select a button that causes the selected images to be sent for analysis. In another embodiment, a set of rules can be configured to automatically send image for analysis based on one or more factors such as the type of device used to generate the images, etc. In either case, when an AI analysis is complete, the user is notified. In one embodiment, a pop-up window will appear with results of the AI analysis. A report may also be generated including the results of the AI analysis. Regulatory information can be included in analysis results and/or reports for convenience.

In one embodiment, AI usage is monitored and reports regarding AI usage can be generated for review.

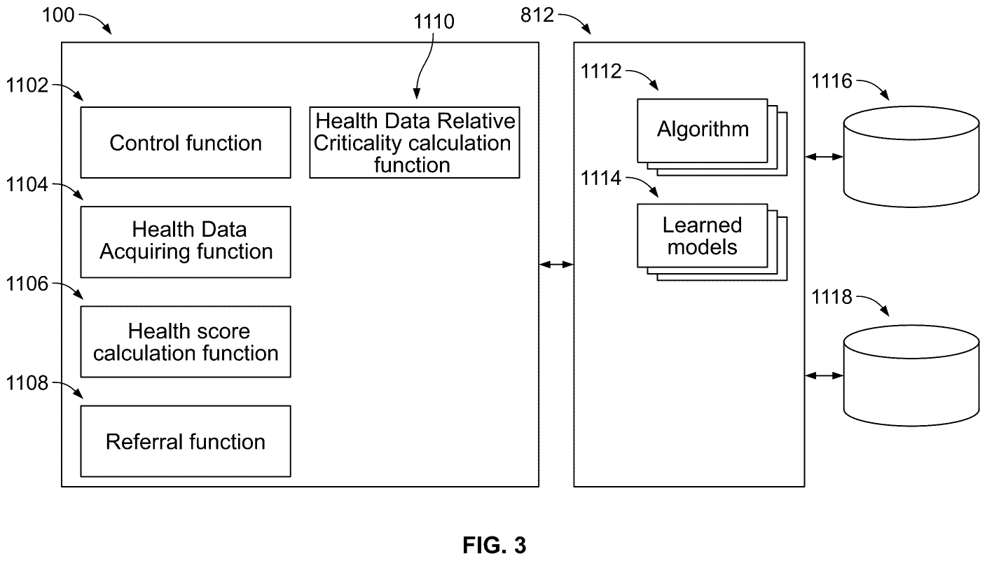

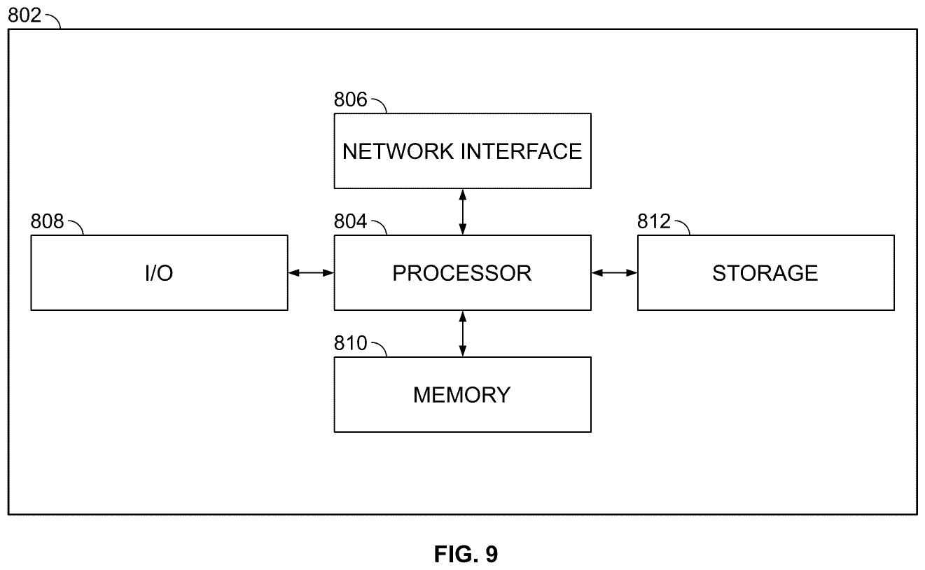

FIG. 3 shows clinical decision support system 100 according to one embodiment. CDSS 100 (shown in FIGS. 1 and 2) comprises control function 1102. In one embodiment, control function 1102 is a function that performs flow control of the functions/steps including selection of algorithms, determining if the other algorithms are required based on the health score of a first algorithm, and display control to display a score, or scores, and health data. CDSS 100 also includes health data acquiring function 1104 for acquiring health data and health score calculation 1106 which, in one embodiment, calculates health scores. Referral function 1108 is included in CDSS 100 and performs operations associated with referrals. In one embodiment, CDSS 100 includes health data relative criticality calculation function 1110 which performs calculations pertaining to health data relative criticality. CDSS 100 is shown in communication with storage 812 (shown in FIG. 9 and described below). In one embodiment, storage 812 stores algorithms 1112 (e.g., algorithms for generating scores based on examination data) and learned models 1114. In one embodiment, learned models 1114 are used for calculating the score utilizing machine learning (e.g., AI). The learned models 114 are generated by inputting a certain amount of the health data of patients with a specific disease (for example, glaucoma in this embodiment) and applying a supervised learning algorithm, such as a Support Vector Machine (SVM). In one embodiment, the generated learned model is used to output the likelihood (classification accuracy) of the specific disease when the learned model is supplied with new health data of a patient to be screened. The present disclosure does not particularly limit the machine learning algorithm and learned models 1114 represents the calculation of the score by employing existing AI technologies. In one embodiment, storage 812 is also in communication with patient database 1116 which stores patient information and normative database 1118 which stores normative data.

FIG. 4 shows method 300 operated by CDSS 100 for allowing a user to display, review, and manage patient health data and for providing a score associated with a patient according to an embodiment. In one embodiment, control function 1102 of CCDSS 100 shown in FIG. 3 controls operation of CDSS 100. In one embodiment, health data, including examination parameters, are used together with an algorithm to generate a score. In this embodiment, an artificial intelligence algorithm is used to generate the score, but other types of algorithms can be used as well.

Method 300 begins at step 302 where health data associated with a patient is received. In one embodiment, health data is received by health data acquisition function 1104 of CDSS 100 shown in FIG. 3. The health data can include, for example, examination parameters obtained from an examination using devices such as shown in FIGS. 1 and 2. In one embodiment, the health data is received by CDSS 100 from data management software 206. The health data received from data management software 206 can be received by data management software 206 from one or more sources. For example, health data can be acquired by one or more of OCT device 102, slit lamp 106, and visual field analyzer 110 and transmitted to data management software 206 via integration service 214 and SSL DICOM interface 208. Health data may also be received by data management software 206 from user 220 via HTTPS user interface 218 and by user 220 entering data into browser 222. Health data may also be received by data management software 206 from user 226 (e.g., a remote doctor) via HTTPS user interface 218 by user 226 entering data into browser 228, or from 3^{rd} party EMR 236 via EMR interface 234.

Returning to step 302 of FIG. 4, after health data associated with the patient is received, method 300 proceeds to step 304 where an algorithm is selected to analyze the received health data and generate a score. In one embodiment, health score calculation function 1106 shown in FIG. 3 selects an algorithm to calculate a score (e.g., a health score). In one embodiment, a score indicates a range of risks with respect to a disease. An algorithm can be selected using one of a variety of methods. In one embodiment, the algorithm is selected from a plurality of candidate algorithms (e.g., algorithms available to select) including, for example, multi-factoral optical coherence tomography (OCT) score (MOS, also known as Fukai-Nakano); glaucoma health score; classic ocular hypertension score (OHTS); OCT tension score; or OCT-enhanced OHTS, based on user input. These algorithms are described in further detail below. In one embodiment, the algorithm selected is based on a default setting. The algorithm can also be selected based on the received health data. For example, the type or source of the health data received, including examination parameters, may be conducive to analysis by a particular algorithm. The algorithm can also be selected by a user. In one embodiment, when the color associated with all examination parameters used for a particular algorithm indicates that all examination parameters are within a desired range, but the score is out of range (e.g., a dangerous value) another algorithm will be selected. This is because another algorithm is likely to produce a different score of the user and/or patient to consider.

After the algorithm is selected at step 304, the method proceeds to step 306 where a score associated with the patient is calculated using the received health data associated with the patient and the selected algorithm.

After the score is calculated at step 306, the method proceeds to step 308 where it is determined whether one or more other algorithms should be used based on the calculated score. For example, a score generated using a first algorithm may not be indicative of an issue in a conclusive manner or may be a value that would typically alert a user (e.g., a health care professional) that additional analysis of the received health data associated with the patient is required. In one embodiment, when the color associated with all examination parameters used for a particular algorithm indicates that all examination parameters are within a desired range, but the score is out of range (e.g., a dangerous value) another algorithm will be selected. This is because another algorithm is likely to produce a different score of the user and/or patient to consider. In one embodiment, a user and/or patient may be asked if they want to use an algorithm if the algorithm requires payment to use. If it is determined that other algorithms will not be used, the method proceeds to step 312 and results are displayed (described in detail below). In one embodiment, health score calculation function 1106 of CDSS 100 shown in FIG. 3 performs step 308 shown in FIG. 4 and determines whether to use other algorithms based on the score.

If it is determined that additional algorithms should be used, the method proceeds to step 310 where additional scores are calculated using one or more additional algorithms in response to determining that other algorithms should be used to calculate scores.

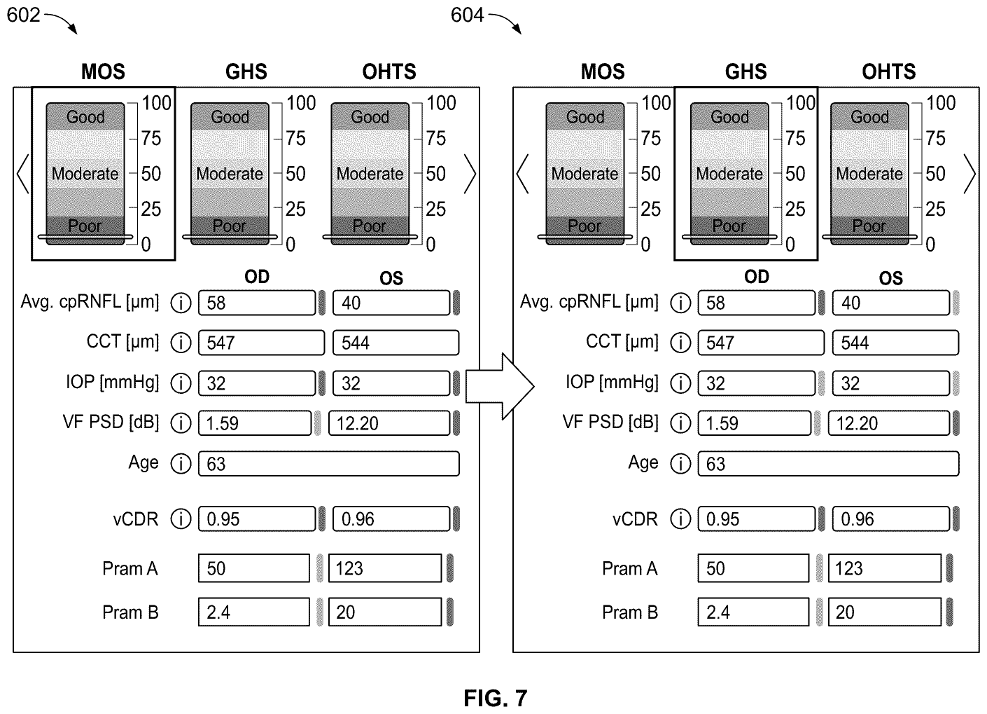

At step 312, the results (i.e., the calculated score or scores) are displayed. In one embodiment, the results are displayed in a report that can include information regarding the received health data and examination parameters. In one embodiment, a report displays scores generated using different algorithms in a single consolidated image/display (see FIG. 7 and associated description). In one embodiment, a report comprises information that is compiled for a user. For example, a report can include information such as examination parameters and the result of analysis of the examination parameters by an algorithm. In one embodiment, a report is an electronic document that can be displayed for a user and printed if a user would like a hard copy.

At step 314, the result(s) are prepared for sharing with other health care professionals. In one embodiment, referral function 1108 of CDSS 100 shown in FIG. 3 perform step 314 and prepares results to be shared with other doctors. In one embodiment, after a user has reviewed the calculated score and verified the assessed factors and examinations, the user can decide what the next steps should be and record their conclusion. The conclusion is saved in a report which can be used, for example, during the patient's next visit or when referring them to a specialist. The report content can include the calculated score, assessed factors, decision(s), and comments. The full content of the report can vary between algorithms. In one embodiment the report can be shared with another eye care professional for their review. In one embodiment, a user can select what next steps are necessary for a patient from a list. In one embodiment, the list includes the options of No Action: the patient shows no signs of increased glaucoma risk, no further actions required at this point; Monitor: there is no need for urgent actions, but the patient's condition should be monitored closely; Refer; Additional Testing: the results show an increased risk of glaucoma and further testing is required; and Consultation. In one embodiment, No Action is the default and will be recorded as the decision if another selection is not made.

The benefits of CDSS 100 include better quality and efficiency of glaucoma testing and services, possible reimbursement for additional testing due to CDSS result indicating medical necessity, reduced medical liability, better efficiency of decision making, better quality of referrals, reduction of over-referrals, and higher patient retention rate.

Details of various embodiments of the displayed results are described as follows. In one embodiment, each algorithm uses a particular group of examination parameters and each of the examination parameters (e.g., factors) may have a different relative criticality (also referred to as criticality). In one embodiment, an indication of the relative criticality of each of the examination parameters is based on a normative database. In one embodiment, the indication of the relative criticality of each of the examination parameters is based on thresholds for abnormality. In one embodiment, the indication of the relative criticality is with respect to an associated score or algorithm. In one embodiment, the relative criticality of factors is shown using colors. In one embodiment, the relative criticality of a parameter is based on how much the particular parameter affects the score generated using a particular algorithm. In one embodiment, the relative criticality values are either percentiles from the Normative Database (e.g., for all parameters extracted from OCT) or established thresholds for abnormality (similar to thresholds for IOP). In one embodiment, relative criticality is determined using the percentile compared to the normative database. In other embodiments, relative criticality is based on a fixed range defined by using the clinical experiences. In one embodiment, relative criticality is based on ranges defined in the algorithms where some examination parameters are defined as risk factors.

In one embodiment, the percentiles and other thresholds are not used or generated by the algorithms. The algorithms are independent of the normative databases and were validated using different populations than the populations used with the normative data.

Scores of a patient can be determined using well-known algorithms such as the OHTS, MOS, and GHS algorithms and the criticality of the examination parameters can be shown using colors. A brief description of these well-known algorithms follows.

The OHTS algorithm is a calculator that uses a point system for estimating a patient's risk for developing a primary open-angle glaucoma (POAG) within five years. The OHTS algorithm is derived from two studies, the Ocular Hypertension Treatment Study and the European Glaucoma Prevention Study. The OHTS algorithm and the clinical validation data are described in: Validated Prediction Model for the Development of Primary Open-Angle Glaucoma in Individuals with Ocular Hypertension, Ophthalmology, Volume 114, Issue 1, 10-19.e2.

The parameters used in the OHTS algorithm are age of patient, vertical cup-to-disc ratio (vCDR), intraocular pressure (IOP), central corneal thickness (CCT), and threshold visual field pattern standard deviation (VF PSD). The averaged circumpapillary retinal nerve fibre layer thickness (Av. cpRNFL) may not be used in the algorithm but relate to glaucoma status and may be of interest to a user.

In one embodiment, scores are calculated and then displayed using a graph, such as a bar graph. A bar graph can show various percentile ranges using different colors. In one embodiment, the calculated score is compared to a normative database and the result of the comparison is a percentile in which the value lies. In one embodiment, a normative database is a collection of data that represents normal or standard values of a particular characteristic or set of characteristics within a specific population. A normative database can be used in medical and scientific fields to compare individual test results with typical or expected values, allowing for the identification of deviations that may indicate a health condition or other anomalies.

Calculating percentiles from a normative database provides a user with an indication of where an individual data point or a sample falls within an overall distribution. One benefit of calculating percentiles from a normative database includes providing a benchmark for individual evaluation. For example, percentiles indicate the relative position of a specific data point within the entire dataset. Evaluating where a patient's measurement falls within the normative database's percentiles can help determine whether the patient is within the normal range or showing abnormal values. Another benefit is the detection of outliers. By using percentiles, it is easier to identify outliers, such as extremely high or low values. This can be crucial for diagnosis and prevention. For instance, if a test result falls in the 95th percentile, it indicates that the individual's value is significantly higher than average, possibly warranting further testing or treatment.

It should be noted that the percentiles are determined by the normative database, which stores normal data for a specific population, and from which, the percentiles are calculated. Different normal data will produce different percentiles. In one embodiment, a normative database is generated by gathering data from a representative sample of a population. Statistical methods are used to establish what is considered “normal” for the group, often defining ranges like the 95th percentile or mean plus/minus standard deviations.

In one embodiment, percentile ranges are defined by a device that was used to generate the percentile data. In one embodiment, intraocular pressure percentile ranges are based on established clinical guidelines. In one embodiment, ranges of percentile values are defined and associated with a color. For example, each range of percentiles is displayed using a color associated with that range. In one embodiment, the information pertaining to the increments used to define the ranges as well as the color codes is stored in a normative database. In one embodiment, the normative database is part of CDSS 100 shown in FIGS. 1 and 2 but can be located elsewhere. The number of increments and the set of colors vary for different OCT thickness parameters and their respective normative databases (e.g., cpRNFL, OCT thickness parameters, OHTS, vCDR, etc. may each have their own normative database). In one embodiment, a normative database is adjusted based on age and optic disc area using, for example, quantile regression. Thus, if the birthdate of a patient is not input or if optic disc area is not available due to, for example, a calculation error, comparison with the reference data is not performed. If age and/or disc area are out of range of the normative database, then percentiles are calculated by extrapolation. In one embodiment, intraocular pressures are established using guidelines. In one embodiment, the percentile related color codes and ranges are fixed for risk factors.

In one embodiment, a bar graph associated with a score related to the OHTS algorithm is displayed using colors representing the estimated 5-year risk of developing primary open angle glaucoma where < or =4% is shown in blue, 5-10% is shown in light blue, 11-15% is shown in light yellow, 16-20% is shown in dark yellow, and > or =33% is shown in red.

The vCDR criticality is shown using colors associated with percentile ranges with 0-1% being red, 1-5% being yellow, and 5-100% being green. In one embodiment, the IOP criticality is shown using colors associated with pressure ranges with IOP>22 mmHg being red, IOP 20-22 mmHg being yellow, and IOP<20 mmHg being colorless. Av. cpRFNL criticality is shown using colors associated with percentile ranges with 0-1% being red, 1-5% being yellow, 5-95% being green, and 95-100% being white.

The multi-factoral optical coherence tomography (OCT) score (MOS, also known as Fukai-Nakano) is an OCT based score for population-based glaucoma mass screening calculated from retinal thickness-related values obtained through spectral domain optical coherence tomography. The description of the algorithm and the clinical validation data are available in the following publication: Fukai, Kota et al. “Real-Time Risk Score for Glaucoma Mass Screening by Spectral Domain Optical Coherence Tomography: Development and Validation.” Translational vision science & technology vol. 11,8 (2022): 8. doi:10.1167/tvst.11.8.8.

MOS is calculated from retinal thickness-related values obtained through spectral domain optical coherence tomography. The parameters used in the algorithm are Av. cpRNFL, and minimum sector thickness value of the 6-sector macular thickness grid for the macular Ganglion Cell Layer+Inner Plexiform Layer (Min. mGCL+). Age, vCDR, IOP, CCT, and VF PSD may not be used in the calculation but relate to glaucoma status and may be of interest to a user. In one embodiment, retina thickness measurements are shown using six colors where 0-1% is shown in red, 1-5% is shown in yellow, 5-95% is shown in green, 95-99% is shown in orange, and 99-100% is shown in magenta.

In one embodiment, a bar graph associated with a score related to the MOS algorithm is displayed using colors representing the estimated glaucoma risk and the need for detailed examination where 0-49% is shown in blue, 50-89% is shown in yellow, and 91-100% is shown in red.

The Av. cpRNFL criticality is shown using colors associated with percentile ranges of a normative database with 0-1% being red, 1-5% being yellow, 5-95% being green, and 95-100% being white. In one embodiment, the Min mGCL+ criticality is shown using colors associated with percentile ranges with 0-1% being red, 1-5% being yellow, and 5-100% being green. In one embodiment, the vCDR criticality is shown using colors associated with percentile ranges with 0-1% percent being red, 1-5% percent being yellow, and 5-100% percent being green. The IOP criticality is shown using colors associated with pressure ranges with IOP>22 mmHg being red, IOP 20-22 mmHg being yellow, and IOP<20 mmHg being colorless.

The Glaucoma Health Score (GHS) is a multi-factoral evaluation in relation to likelihood of having of glaucoma. GHS is a score for population-based glaucoma screening calculated from several parameters, including IOP, CCT, RNFL, GCL, and visual field PSD. The parameters used in the algorithm are age, Av. cpRNFL, Min. mGCL+, IOP, CCT, and VF PSD. vCDR may not be used in the calculation but relates to glaucoma status and may be of interest to a user.

In one embodiment, a bar graph associated with a score related to the GHS algorithm is displayed using colors representing a score range with 0-49% shown in blue, 50-89% is shown in yellow, and 90-100% is shown in red.

The Av. cpRNFL criticality may be shown using colors associated with percentile ranges with 0-1% being red, 1-5% being yellow, 5-95% being green, and 95-100% being white. The Min mGCL+ criticality may be shown using colors associated with percentile ranges with 0-1% being red, 1-5% being yellow, and 5-100% being green. The IOP criticality may be shown using colors associated with pressure ranges with IOP>22 mmHg being red, IOP 20-22 mmHg being yellow, and IOP<20 mmHg being colorless.

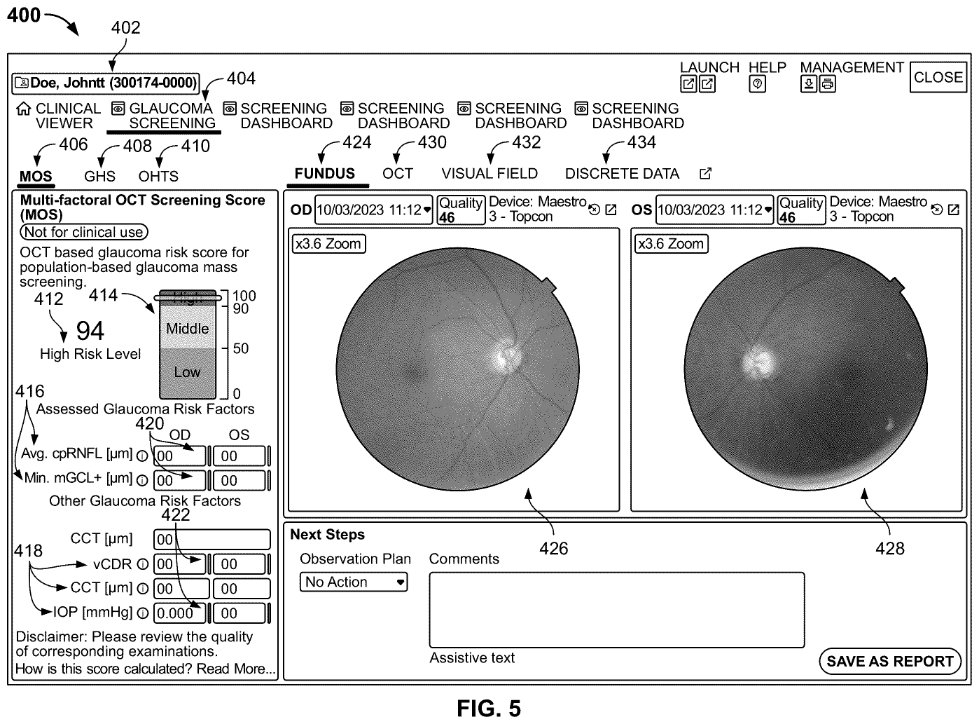

FIG. 5 shows the results (i.e. calculated score or scores) in report 400 displayed as described in step 312 of FIG. 4. Patient name 402 identifies the patient to which report 400 pertains. Glaucoma tab 404 is shown as being selected and indicates that the information displayed pertains to glaucoma screening.

MOS tab 406, GHS tab 408, and OHTS tab 410 can each be selected to view results determined using the related algorithm. MOS 406 tab is shown as being selected and indicates that the results shown in the area below the tab pertain to multi-factoral OCT screening score (MOS) results. GHS 208 tab and OHTS 410 tab can be selected to display those results as desired by a user. It should be noted that a tab will only be shown for an algorithm if it was used to analyze health data and the results shown in response to selecting a tab relate to the algorithm identified on the selected tab.

Score 412 displays the calculated score that is based on the examination parameters and the selected algorithm. In one embodiment, the score is displayed with the color of the score being based on a risk level associated with the score. For example, scores that are associated with low risk can be blue while scores associated with middle level risk can be yellow and scores associated with high level risk can be shown in red. Bar graph 414 graphically displays the calculated score using a horizontal bar located vertically along bar graph 414. Bar graph 414 can be shown having differently colored segments with the colors of the segments indicating the risk level associated with the range of scores represented by the segments. For example, scores that are associated with low risk can be colored blue while scores associated with middle level risk can be colored yellow and scores associated with high level risk can be shown in red. In one embodiment, the color of the score 412 is the same as the color of bar graph 414 at which the horizontal bar is located.

In one embodiment, a patient is referred to another eye care provider if a score exceeds a threshold (also referred to as a cut-off). Each algorithm has a particular threshold for determining whether a referral is required or suggested. For example, the threshold for the Fukai algorithm is set at 90. If a patient has a Fukai score of 90 or higher, the patient should be referred to another eye health care provider, such as a specialist. Scores can be modified based on additional information, such as known false negatives occurring using a particular score. For example, if a particular number of false negatives occurred using a threshold score of 90, the threshold score can be lowered in order to reduce or eliminate false negatives. In one embodiment, a marker is used to indicate a threshold value. For example, a dashed line can be used with a graph, such as a bar graph, to indicate the threshold value. In one embodiment, a user can adjust the threshold value and that threshold value can be associated with one or more of an algorithm, a user, a patient, an eye care professional, or other person or entity. For example, a threshold can be associated with an algorithm. A threshold can also be associated with an eye care professional who does not want to see patients having a score exceeding the eye care professional's desired threshold.

Examination parameters including assessed risk factors and other risk factors are displayed below score 412 and graph 414. The assessed risk factors 416 that are displayed are assessed glaucoma risk factors. Below assessed risk factors 416, other risk factors 418 are displayed, in this case, other glaucoma risk factors. Relative criticality 420, 422 of each of the examination parameters with respect to the related score can be represented using different colors associated with each of the examination parameters. The risk factors shown are based on the particular algorithm being used. For example, FIG. 4 shows risk factors associated with the MOS algorithm in response to MOS tab 406 being selected. In one embodiment, a sort function can be used to organize how risk factors are displayed below a score. For example, critical parameters can be located at the top of the list of parameters located below the score 412 and graph 414.

Measurement data (e.g., images and or text) associated with examination parameters are shown on the right side of report 400. Measurement data associated with examination parameters are obtained using one or more devices (e.g., one of more of devices 102 through 112 shown in FIG. 1). Fundus tab 424 indicates that the examination parameters shown are fundus images 426, 428. In one embodiment, fundus images are displayed for review by a user (e.g., an eye care provider) and are not analyzed to produce a score or other data. Other tabs can be selected to show other data associated with examination parameters such as OCT tab 430, visual field tab 432, and discrete data 434, for example. In one embodiment, the data shown on the right side of report 400 pertains to the examination parameters that were used by the selected algorithm to generate score 412. In other embodiments, the data shown on the right side of report 400 can pertain to any examination parameters regardless of whether they are associated with the selected algorithm.

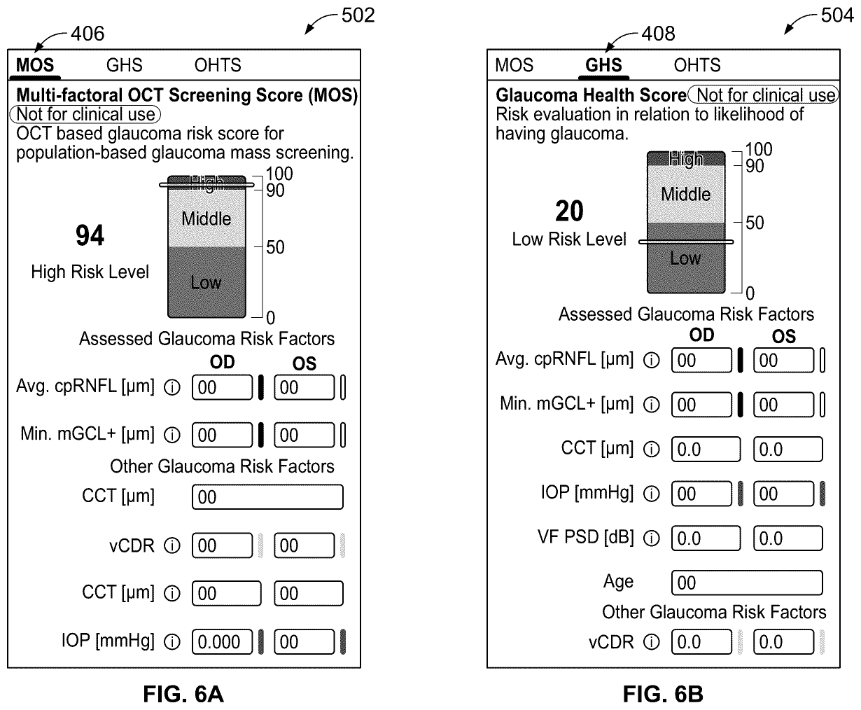

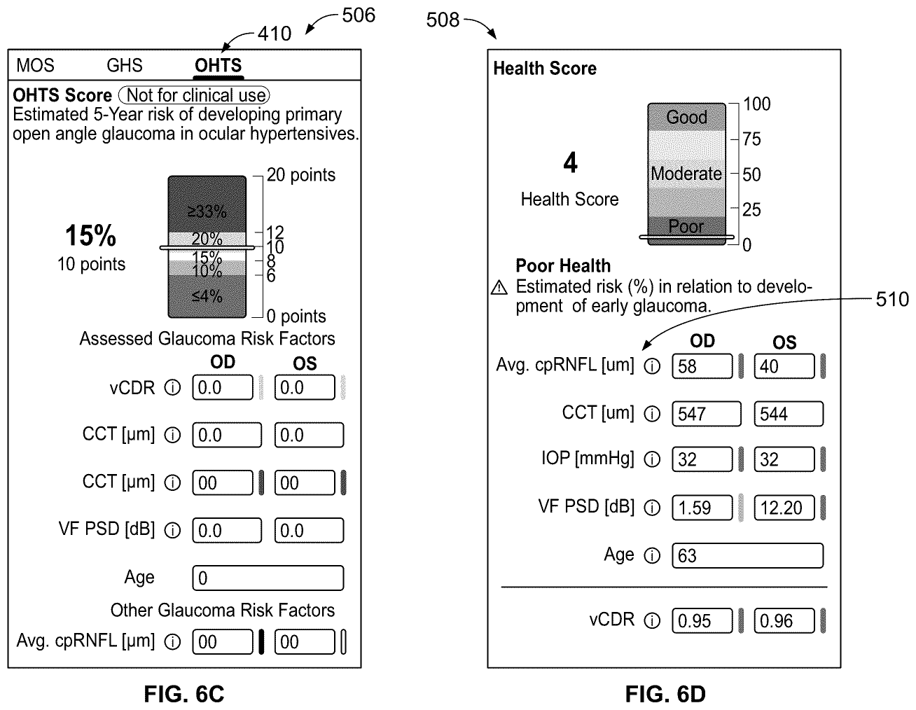

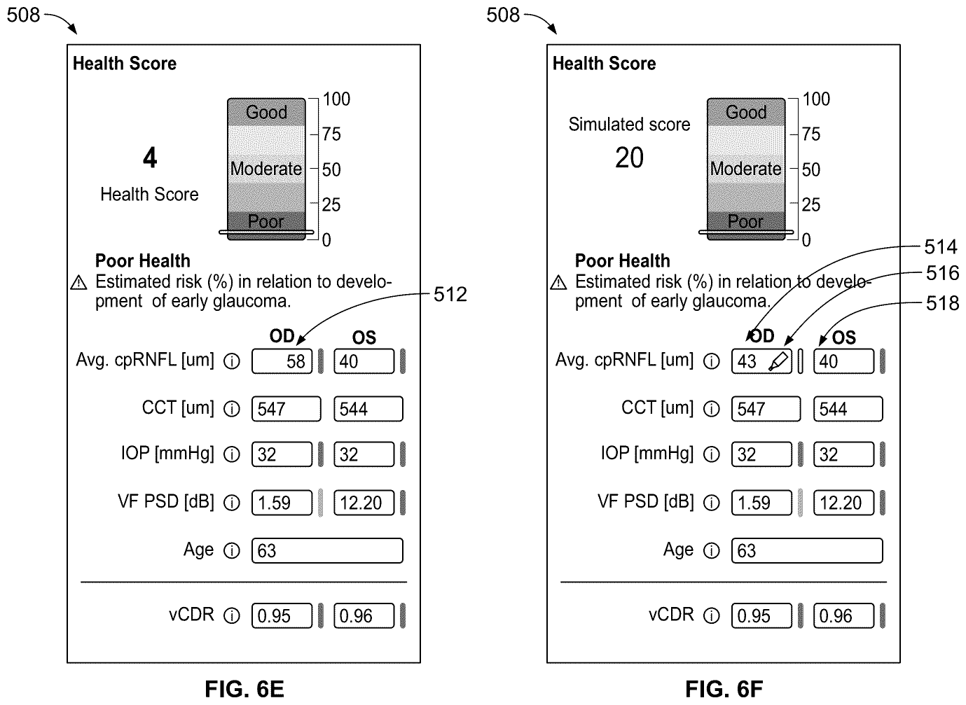

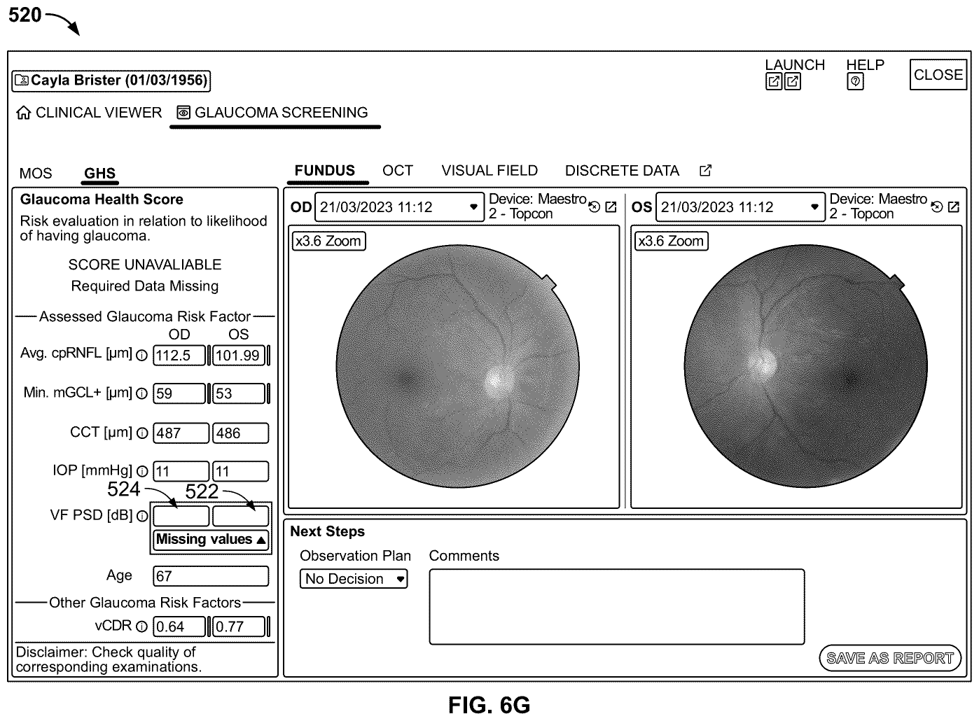

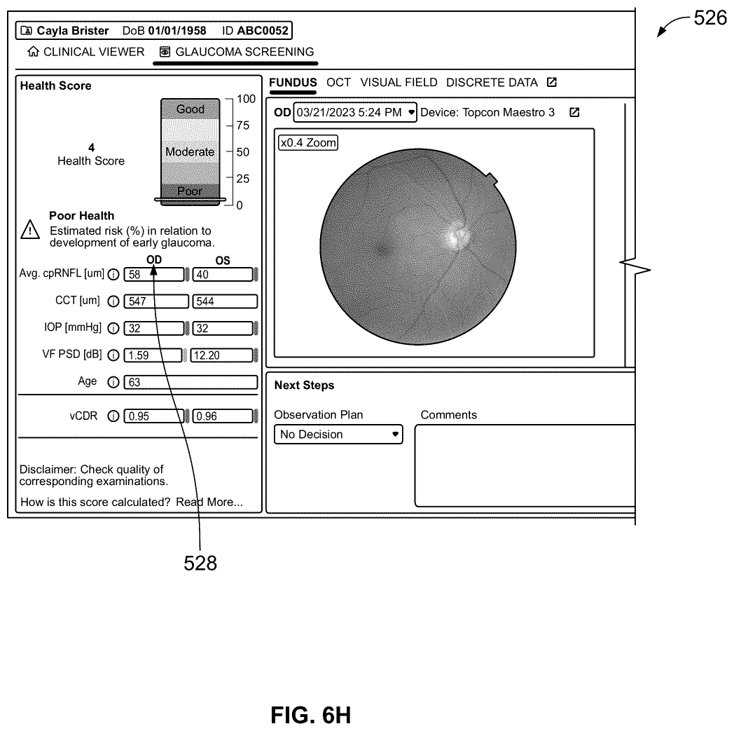

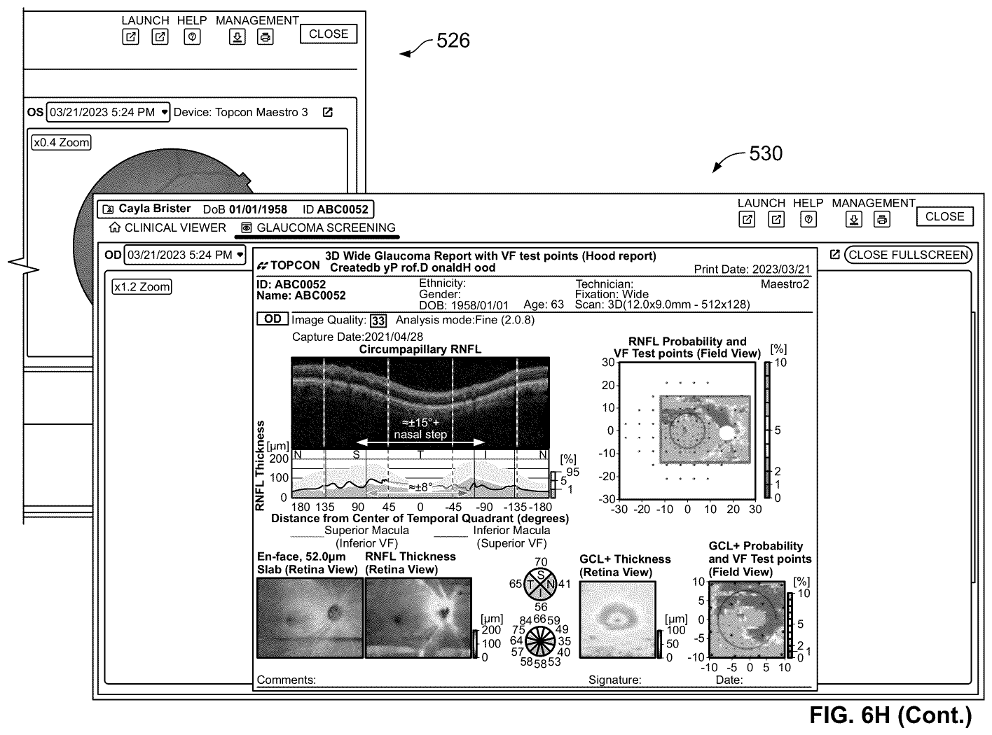

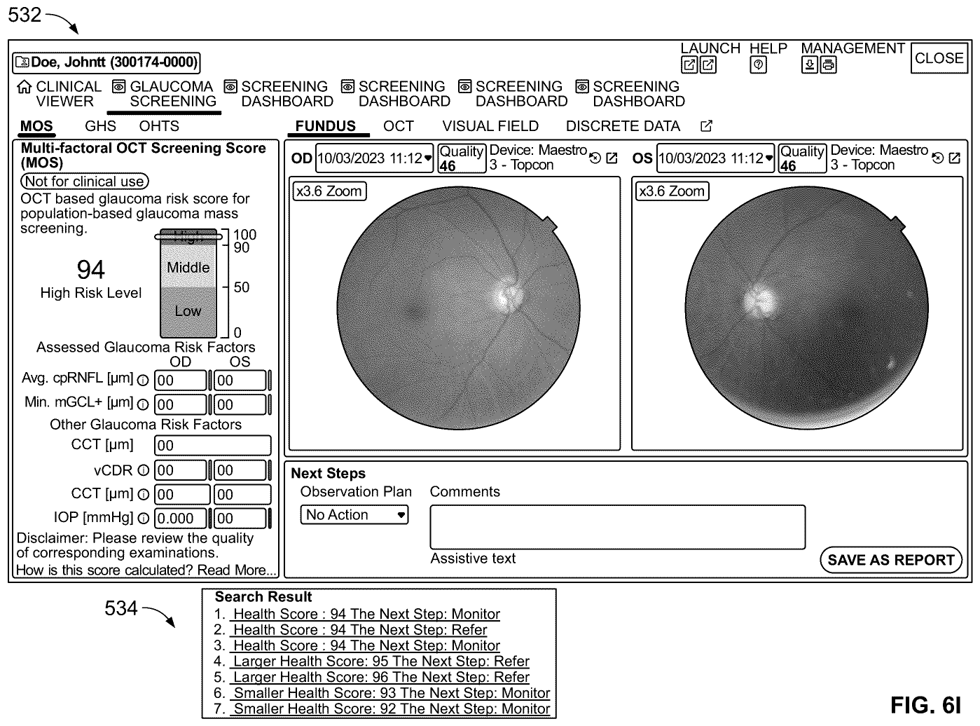

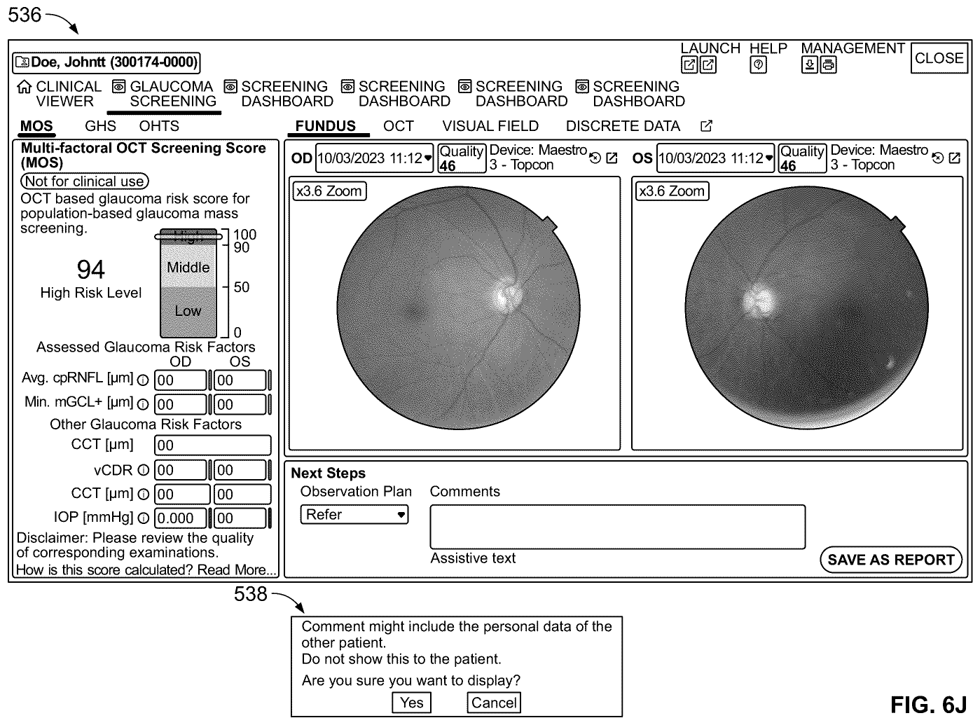

FIGS. 6A-6C show the scores and examination parameters that are displayed in report 400 in response to one of tabs MOS 406, GHS 408, and OHTS 410 being selected. For example, the image shown in FIG. 6A is displayed on the left side of report 400 when MOS tab 406 is selected. Similarly, the image shown in FIG. 6B is displayed on the left side of report 400 when GHS tab 408 is selected. The image shown in FIG. 6C is displayed on the left side of report 400 when OHTS tab 410 is selected.

It should be noted that in one embodiment, parameters can be changed by a user manually entering parameter values (referred to as a health data re-entry). A score determined based on the parameter will be updated with a new value after a parameter has been changed by a user. FIG. 6D shows display 508 in which a user can select parameter 510. After selection of parameter 510, text box 512 opens with the current value of the examination parameter (in this case “58”) is displayed as shown in FIG. 6E. A user can then change the examination parameter (from “58” shown in FIGS. 6D and 6E to “43” shown in FIG. 6F) by entering a new value. As shown in FIG. 6F new value 514 is displayed differently from old values. Indicator 516 is displayed showing that the examination parameter has been changed and color indicator 518 also changes to display the relative criticality of the new value. In one embodiment, an examination parameter that has been changed may be identified by different colored text, a different colored background, and/or an icon displayed next to the changed value. In one embodiment, examination parameter values are entered when one or more examination parameters are not available from examinations. In another embodiment, a user can change an examination parameter in order to see the effect of a change of a parameter in the future may affect a patient's score or relative criticality of an examination parameter. In one embodiment, a score is displayed in a way that indicates that it is based on one or more values entered by a user. For example, the color of the score or bar graph may be changed, or a descriptive text or icon may be displayed near the score indicating that the score is based on one or more values entered by a user. In one embodiment, a reset button is provided in order to reset the changes entered by a user. When a user enters a value for any health data, a reset button can be displayed next to the changed value. When the user presses the reset button, the value displayed returns to its original value. The associated score will be recalculated using the original values and displayed in response to selection of the reset button.

This health data re-entry feature allows users to simulate the impact of health data (e.g., examination parameters) on scores. For example, this can be used when a particular score is not displayed due to missing patient health data. Another example is when a patient's score is in the yellow area, for example, and the user wants to see how much the health data should be changed to make the patient's score reach the red area. However, since a score based on user re-entered values is not based on data measured by diagnostic devices, it is more appropriate to indicate that it is based on what the user has re-entered.

FIG. 6G shows a display 520 in which input boxes 522 and 524 are provided for a user to enter missing health data. After the user enters the missing health data, a score and graph will be displayed.

In one embodiment, a user can click on an examination parameter in order to display data related to that examination parameter. FIG. 6H shows display 526 in which a user selects examination parameter 528. In response to the selection of examination parameter 528, display 530 is displayed to the user. In one embodiment, when a user selects any health data (e.g., examination parameters) examination data is displayed to the user based on the device used to determine the examination parameter. For example, if the health data is vCDR, a Fundus image is displayed, if the health data is IOP, discrete data pertaining to IOP is displayed, and if VF PSD is selected, a visual field is displayed. A benefit of this function is that data related to health data is displayed in response to selection of health data thereby allowing easy access to additional data. This functionality allows users to view the data related to the health data of the user without the user having to select a tab, thereby making it easier for the user to refer to the data. When using the health data re-entry function, which is described above, this functionality allows the user to refer to the value of the health data to be re-entered without moving mouse focus.

In one embodiment, a function is available for displaying scores and examination parameters of other patients with similar health data. In one embodiment, other patients' data, including score and examination parameters, can be viewed for reference. Because the user needs to determine the next action to be taken with respect to a patient, the actions taken for other patients having similar scores and examination parameters can be viewed to aid the user in determining what action should be taken next. Since patient data is sensitive, data that can be used to identify a particular patient having similar scores and examination parameters can be hidden from view. In one embodiment, any data that may contain personal or sensitive data is hidden and requires separate and additional action to be taken by a user to view that data. In one embodiment, the “Next Steps” section (or “Next Actions”) includes an “Observation plan” section and a “Comments” section, where the observation plan has some options including “No action”, “Monitor”, “Refer” and “Consultation” and content of Comments (500-character text data). In one embodiment, personal information in the comments section is hidden to prevent disclosure of a patient's personal information. In one embodiment, the observation plan is referred to by a user who want to see the other patient's data. In one embodiment, a button may be displayed to allow a user to push the button to view the other patient's data. In one embodiment, patients having a score within the range of approximately ±2-3 or examination parameters within a range about a patient's examination parameters will be selected for display. FIG. 6I shows display 532 in which a user has selected an option to view other patient's data. In response, display 534 including a list of hyperlinks of patients having similar scores that can be viewed by the user. The user can select each hyperlink to view the related information.

In one embodiment, a user must take an additional action in order to view sensitive data, such as comments related to a patient. FIG. 6J shows display 536 in which a user has selected to view comments regarding a patient having a score and/or examination parameters similar to a patient being examined. In response to selecting to view comments, pop-up window is displayed to a user having the text “Comment might include the personal data of other patient. Do not show this to the patient. Are you sure you want to display?” along with “Yes” and “Cancel” buttons which allow the user to view the data or cancel their request.

The functionality described above allows a user to consider the next step for the patient with reference to the data of other patients. The user is often unsure of the next action to take when the score is inconclusive, but this feature allows the user to refer to past data determined by other clinics' doctors and opticians. In consideration of displaying other patients' data, the system controls that some information is not displayed, and some information requires an action before it can be displayed.

In one embodiment, multiple scores and examination parameters can be consolidated and displayed in a single image. FIG. 7 shows the scores associated with MOS, GHS, and OHTS displayed consolidated into a single image 602. Particular examination parameters and their relative criticality with respect to the currently selected score are shown below the bar graphs. As previously described, the relative criticality of each of the examination parameters are shown colored with each color representing a different range of criticality values. Consolidated image 602 shows particular examination parameters and their relative criticality below the bar graphs. The particular examination parameters shown are based on which one of the three bar graphs is selected. For example, consolidated image 602 shows examination parameters and their criticality associated with the MOS algorithm (shown enclosed in a rectangle in response to the related bar graph being selected). The examination parameters and criticality associated with other algorithms can be displayed by selecting a bar graph associated with a different algorithm. Consolidated image 604 shows the GHS algorithm selected (shown as selected by being enclosed in a rectangle) and the examination parameters and criticality associated with the GHS algorithm are displayed in response to the selection.

In one embodiment, a user can request (e.g., by selecting a related icon) to be shown examination parameters and images of other patients which have the same or similar examination parameter values. Several screening results of other patients having the same score can be displayed to a user in response to the request. This additional information can be used to aid in determining a course of action for a patient. For example, when a score of a patient displayed to a user is in a “yellow” range, meaning that the value is not out of range but close to being out of range, a user may not know whether to refer the patient to another eye care professional. By displaying examination parameters and scores for patients having similar examination parameters and scores, a user can review whether other patients with similar scores were referred to eye care professionals and determine whether their current patient should be similarly referred.

In one embodiment, scores for a patient determined during a current visit can be displayed next to scores for the same patient determined at an earlier date and time. This can allow a user to see changes in scores for a patient over time. In one embodiment, when no prior scores or examination data is available for a patient, the next visit for a patient can be estimated or predicted based on the examination parameters determined for a patient during a current visit. For example, it may be desirable to perform certain examinations periodically, such as every 3 months, 6 months, or one year. In such cases, the next visit for a patient can be the earliest date for a follow up visit in order to obtain additional examination parameters. In one embodiment, the most critical score determined for a patient during a visit is used to determine when the next visit should be scheduled.

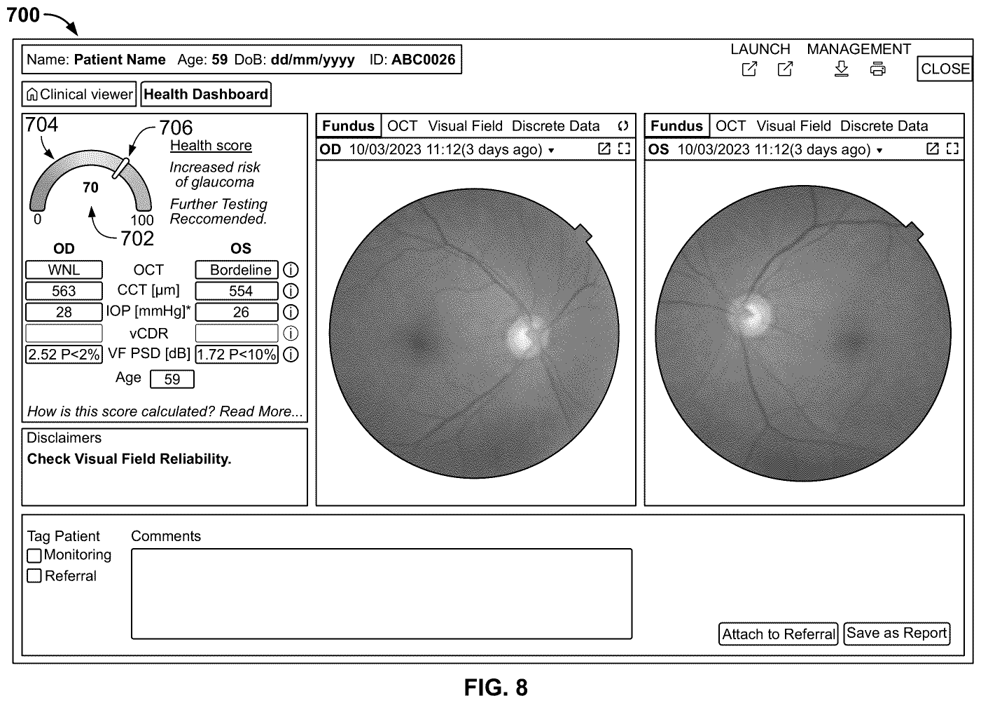

FIG. 8 shows report 700 which is similar to report 400 shown in FIG. 5 with some changes. Score 702 is shown as a numerical value in a manner similar to the display of score 412 shown in FIG. 5. However, instead of bar graph 414 as shown in FIG. 5, report 700 displays a gauge-type chart 704 that graphically shows the value of score 702. Needle 706 is used to indicate where on gauge type chart 704 a value is located. Other types of graphs and/or charts can be used as well and the graphs/charts can be colored to associate the scores with risk values or other indicators.

CDSS 100 shown in FIG. 1, as well as other devices described herein, can be implemented using one or more computers. In addition, the hardware identified in FIG. 2 by a bold solid line border as shown in legend 201 of FIG. 2 can also be implemented using one or more computers. A high-level block diagram of such a computer is illustrated in FIG. 9. Computer 802 contains a processor 804 which controls the overall operation of the computer 802 by executing computer program instructions which define such operation. The computer program instructions may be stored in a storage device 812, or other computer readable medium (e.g., magnetic disk, CD ROM, etc.), and loaded into memory 810 when execution of the computer program instructions is desired. Thus, the method steps of FIG. 4, the software functions (see legend 201) of FIG. 2, as well as other methods and algorithms described herein, can be defined by the computer program instructions stored in the memory 810 and/or storage 812 and controlled by the processor 804 executing the computer program instructions. For example, the computer program instructions can be implemented as computer executable code programmed by one skilled in the art. Accordingly, by executing the computer program instructions, the processor 804 executes an algorithm defined by the method steps of FIG. 4, the software functions of FIG. 2, or other methods and algorithms described the computer 802 also includes one or more network interfaces 806 for communicating with other devices via a network. The computer 802 also includes input/output devices 808 that enable user interaction with the computer 802 (e.g., display, keyboard, mouse, speakers, buttons, etc.) One skilled in the art will recognize that an implementation of an actual computer could contain other components as well, and that FIG. 9 is a high-level representation of some of the components of such a computer for illustrative purposes.

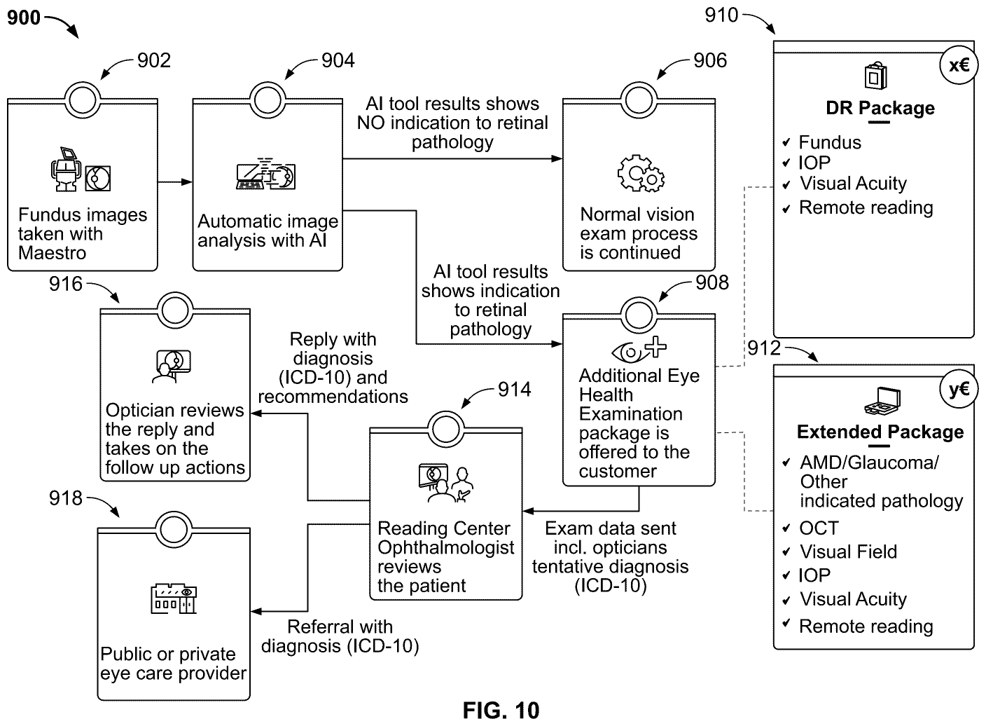

In one embodiment, AI can be used by opticians, primary care providers (such as a generally practitioner), or other user performing eye scans and assist the user in convincing a patient to agree to further examinations based on the results of the patient's eye health exam. FIG. 10 shows a method 900 for opticians to use AI in order to assist in performing a patient's eye health exam. At step 902, fundus images are generated by examination equipment. At step 904, automatic image analysis is performed using AI. If the AI analysis results show no indication of retinal pathology, then the method proceeds to step 906 where the normal vision exam process continues. If the AI analysis results show an indication of retinal pathology, the method proceeds to step 908 and an additional eye health examination package is offered to the patient. For example, package 910 including exams pertaining to fundus, IOP, visual acuity, and remote reading in various tests (e.g., refraction, visual field, color vision, fundus examination, fundus three-dimensional image analysis (OCT), corneal shape, intracorneal capsular examination, deep vision, contrast examination, slit-lamp microscopy, intraocular pressure test, etc.) may be offered to the patient. Extended package 912 includes exams pertaining to AMD/glaucoma/other indicated pathology, OCT, visual field, IOP, visual acuity, and remote reading in various tests may also be offered to the patient. Examination parameters/data and an optician's tentative diagnosis are the sent to a reading center as shown in step 914 where an ophthalmologist reviews the examination parameters/data and other patient information. The ophthalmologist can reply with a diagnosis and recommendations which, at step 916, the optician can review and perform follow up examinations and/or actions. The ophthalmologist can alternatively reply with a referral to another public or private eye care provider (e.g., another ophthalmologist) as shown in step 918 along with a diagnosis.

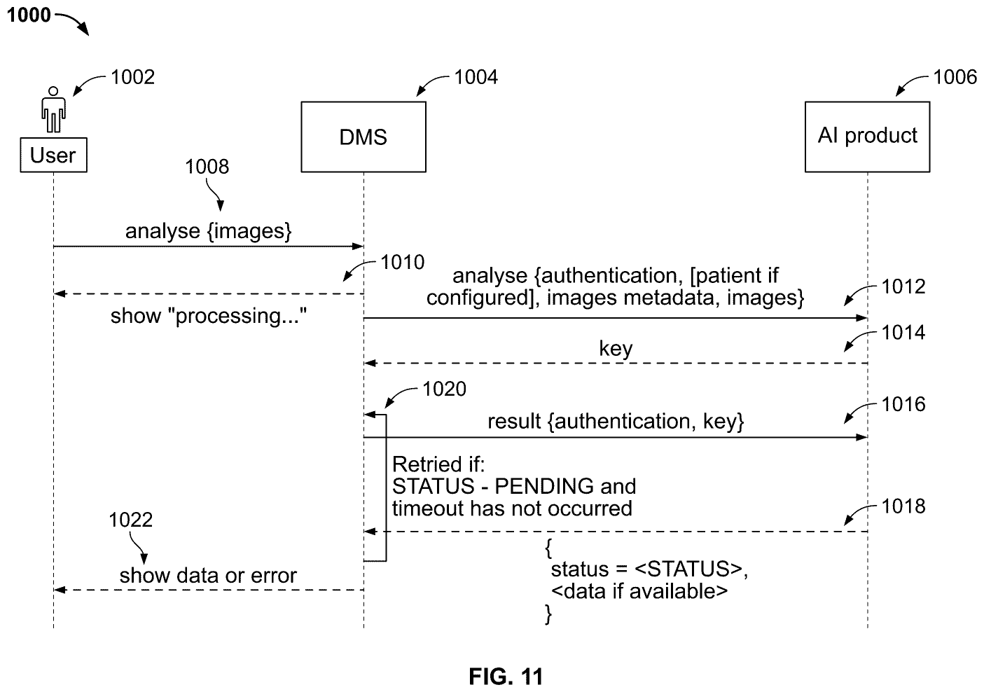

FIG. 11 shows data flow 1000 for image analysis indicating how data flows among user 1002, data management software 1004 (shown as DMS in FIG. 11), and AI product 1006 (e.g., AI that is used to analyze images and/or examination data). At step 1008, user 1002 transmits a request to analyze images to data management software 1004. In response, at step 1010, data management software 1004 transmits an indication to display “Processing . . . ” to user 1002. At step 1012, data management software 1004 transmits a request to AI product 1006 to analyze images in response to the user request of step 1008. The request at step 1012 includes authentication data, patient information (if data management software 1004 is configured to send patient information), image metadata, and images. AI product 1006, at step 1014, transmits a key to data management software 1004. At step 1016, in response to receipt of the key in step 1014, data management software 1004 transmits a result including authentication and the key of step 1014. At step 1018, in response to receipt of the result of step 1016, AI product 1006 transmits the status of its operation or the data that results from the analysis if available. If a result is not available in step 1018, the data flow returns to step 1016 and repeats. At step 1022, the data of step 1018 is transmitted to user 1002 or an error message, if there was a problem with the analysis.

The foregoing Detailed Description is to be understood as being in every respect illustrative and exemplary, but not restrictive, and the scope of the inventive concept disclosed herein is not to be determined from the Detailed Description, but rather from the claims as interpreted according to the full breadth permitted by the patent laws. It is to be understood that the embodiments shown and described herein are only illustrative of the principles of the inventive concept and that various modifications may be implemented by those skilled in the art without departing from the scope and spirit of the inventive concept. Those skilled in the art could implement various other feature combinations without departing from the scope and spirit of the inventive concept.

1. A method for providing a score associated with a patient, the method comprising:
receiving health data associated with a patient, the health data including examination parameters;
calculating a score associated with the patient using an algorithm to analyze the received health data, the score being indicative of a likelihood of one or more target diseases; and
displaying a report comprising the score and an indication of a relative criticality for each of the examination parameters,
wherein:
for each examination parameter, the relative criticality is calculated based on an extent to which each respective examination parameter deviates from at least one respective reference standard associated with the one or more target diseases; and
the indication is a discrete indication generated by associating the calculated relative criticality to one of a plurality of discrete ranges defined by the at least one respective reference standard.

2. The method of claim 1, wherein the indication of the relative criticality of each of the examination parameters is determined based on percentiles calculated from a normative database, wherein the normative database comprises normal-eye data obtained from a selected population diagnosed as free of ocular disease, and provides population-based reference values from which percentiles are calculated.

3. The method of claim 1, wherein the at least one respective reference standard is used to determine whether an individual value of the examination parameter is within a normal range or an abnormal range.

4. (canceled)

5. The method of claim 1, wherein the indication is provided by a color corresponding to one of the plurality of discrete ranges.

6. The method of claim 1, further comprising:
selecting the algorithm from a plurality of candidate algorithms based on the examination parameters associated with the algorithm.

7. The method of claim 1, further comprising:
calculating a second score associated with the patient using a second algorithm to analyze the received health data in response to determining that the examination parameters are within a desired examination parameter range and the score is out of a desired score range; and
displaying a second report comprising the second score and an indication of the relative criticality of each of the examination parameters with respect to the second score.

8. The method of claim 1, further comprising:
receiving user input modifying one of the examination parameters; and
updating the score in response to the user input.

9. The method of claim 1, further comprising:
receiving user input selecting one of the examination parameters; and
displaying data related to the selected one of the examination parameters in response to the user input.

10. The method of claim 1, further comprising:
receiving user input requesting scores and examination parameters of other patients having health data similar to the patient; and
displaying the other patients' scores and examination parameters in response to the user input.

11. An apparatus comprising:
a processor; and
a memory to store computer program instructions for providing a score associated with a patient, which, when executed on the processor cause the processor to perform operations comprising:
receiving health data associated with a patient, the health data including examination parameters;
calculating a score associated with the patient using an algorithm to analyze the received health data, the score being indicative of a likelihood of one or more target diseases; and
displaying a report comprising the score and an indication of a relative criticality of each of the examination parameters,
wherein:
for each examination parameter, the relative criticality is calculated based on an extent to which each respective examination parameter deviates from at least one respective reference standard associated with the one or more target diseases; and
the indication is a discrete indication generated by associating the calculated relative criticality to one of a plurality of discrete ranges defined by the at least one respective reference standard.

12. The apparatus of claim 11, wherein the indication is provided by a color corresponding to one of the plurality of discrete ranges.

13. The apparatus of claim 11, the operations further comprising:
selecting the algorithm from a plurality of candidate algorithms based on the examination parameters associated with the algorithm.

14. The apparatus of claim 11, the operations further comprising:
calculating a second score associated with the patient using a second algorithm to analyze the received health data in response to determining that the examination parameters are within a desired examination parameter range and the score is out of a desired score range; and
displaying a second report comprising the second score and an indication of the relative criticality of each of the examination parameters with respect to the second score.

15. The apparatus of claim 14, the operations further comprising:
displaying the report and the second report together and displaying an indication of the relative criticality of each of the examination parameters with respect to the score or the second score depending upon a user selection.

16. The apparatus of claim 14, the operations further comprising:
consolidating the score and the second score for display in an image.

17. The apparatus of claim 11, wherein the algorithm is an artificial intelligence algorithm.

18. The apparatus of claim 11, the operations further comprising:
receiving user input modifying one of the examination parameters; and updating the score in response to the user input.

19. The apparatus of claim 11, the operations further comprising:
receiving user input selecting one of the examination parameters; and
displaying data related to the selected one of the examination parameters in response to the user input.

20. A computer readable medium storing computer program instructions for providing a score associated with a patient, which, when executed on a processor, cause the processor to perform operations comprising:
receiving health data associated with a patient, the health data including examination parameters;
calculating a score associated with the patient using an algorithm to analyze the received health data; and
displaying a report comprising the score and an indication of a relative criticality of each of the examination parameters with respect to the score,
wherein:
for each examination parameter, the relative criticality is calculated based on an extent to which each respective examination parameter deviates from at least one respective reference standard associated with one or more target diseases; and
the indication is a discrete indication generated by associating the calculated relative criticality to one of a plurality of discrete ranges defined by the at least one respective reference standard.

21. The method of claim 1, further comprising:
receiving user input associated with a conclusion regarding next steps for the patient; and
storing the report including the conclusion,
wherein the conclusion includes a referral to a specialist or another health care professional.

22. The method of claim 21, further comprising:
receiving user input requesting reports of other patients having scores similar to the patient; and
displaying the reports of the other patients in response to the user input,
wherein the reports of the other patients include at least scores and conclusions regarding next steps, and information identifying the other patients is hidden when displaying reports of the other patients.

23. The method of claim 22, further comprising:
receiving user input selecting one of the reports of the other patients; and
displaying a detailed report of the selected one of the reports of the other patients after receiving additional user confirmation indicating that the detailed report includes the information identifying the patient associated with the selected one of the reports of the other patients.

24. The method of claim 2, wherein the percentiles provide an indication of where an individual value of each examination parameter falls within an overall distribution.

25. The method of claim 24, wherein the percentiles are applied to determine whether the individual value falls within a normal range or exhibits an abnormal value warranting further clinical attention.

26. The method of claim 2, wherein the selected population is selected based on patient age.

27. The method of claim 5, wherein relative criticality values in the 0-1% range are shown in red, relative criticality values in the 1-5% range are shown in yellow, relative criticality values in the 5-95% range are shown in green, and relative criticality values in the 95-100% range are shown in orange.

28. The method of claim 8, further comprising:
displaying an indicator adjacent to the modified one of the examination parameters to identify that the modified one of the examination parameters has been modified by the user; and
displaying the updated score in a manner that indicates that the updated score is based on one or more examination parameter values modified by the user.

29. The method of claim 26, further comprising:
displaying a reset button adjacent to the modified one of the examination parameters; and
resetting the modified one of the examination parameters to its value prior to modification in response to user input selecting the reset button.

30. The method of claim 10, further comprising:
receiving user input selecting one of the reports of the other patients; and
displaying a detailed report of the selected one of the reports of the other patients after receiving additional user confirmation indicating that the detailed report includes information identifying the patient associated with the selected one of the reports of the other patients.
